(19) United States
(12) Patent Application Publication
(10) Pub. No.: US 2024/0358295 A1
(43) Pub. Date: Oct. 31, 2024

(54) Blood Draw Device and Method of Use Thereof for a Multi-Site Paired Blood Draw

(71) Applicant: Becton, Dickinson and Company, Franklin Lakes, NJ (US)

(72) Inventors: Prasad Govindaraj, Coimbatore (IN); Jonathan Karl Burkholz, Salt Lake City, UT (US); Sridhaar Nandakumar, Chennai (IN); Ashwin Antony, Trichy (IN); S. Ray Isaacson, Layton, UT (US)

(21) Appl. No.: 18/646,142

(22) Filed: Apr. 25, 2024

Related U.S. Application Data
(60) Provisional application No. 63/461,923, filed on Apr. 26, 2023.

(51) Int. Cl. A61B 5/15

(52) U.S. Cl. CPC A61B 5/150061; A61B 5/15003

(57) ABSTRACT

Provided herein is a blood draw device including a housing defining a housing lumen and an inner volume proximal to the housing lumen, a coupling device, a catheter tube movably received within the housing, and an advancement member movable relative to the housing to move the catheter tube between a first position where the catheter tube is disposed within the housing and a second position where a distal end of the catheter tube is disposed beyond the distal end of the housing. A first port is provided on the housing, in fluid communication with the housing lumen, and a second port is provided proximally from the housing, in fluid communication with the catheter tube. A seal is positioned in the housing lumen proximal to the first port, with the catheter tube proximal to the seal when in the first position and extending through the seal when in the second position.

### CROSS-REFERENCE TO RELATED APPLICATION

The present application claims priority to U.S. Provisional Application No. 63/461,923, entitled “Blood Draw Device and Method of Use Thereof for a Multi-Site Paired Blood Draw” filed Apr. 26, 2023, the entire disclosure of which is hereby incorporated by reference.

### BACKGROUND OF THE INVENTION

### Field of the Invention

The present disclosure relates generally to blood draw devices for use with intravenous (IV) catheters and, more specifically, to blood draw devices that enable a multi-site, paired blood draw via an indwelling IV catheter.

### Description of Related Art

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

An exemplary use of a VAD as described above is in the obtaining of a blood sample for the purpose of performing a blood culture. The blood culture tests for like bacteria or other microorganisms in the blood for purposes of detecting bacteremia (i.e., a blood infection). In performing such a blood culture, it is recognized that it is desirable to obtain multiple blood samples for testing, as a single blood culture may provide a lower sensitivity to detect bacteremia, as well as difficulty in differentiating contaminants from clinically significant pathogens. Accordingly, a paired blood culture is preferred over a single blood culture, as the paired blood culture provides a higher microbiologic yield, along with an improved sensitivity to detect pathogens and time to positivity (TTP).

While it is recognized that a paired blood culture is preferred over a single blood culture, paired blood cultures require the obtaining of two different blood samples at two different sites or locations of the vasculature. Current products in the market allow clinicians to collect blood from one site for the blood culture through the indwelling VAD; however, collection of blood from another site currently requires clinicians to puncture the vein at another location in order to acquire the second blood sample. This need to puncture the vein at another location leads to increased discomfort for the patient and a prolonged procedure time.

Accordingly, it is desired to provide a blood draw device useable with an indwelling VAD that allows clinicians to draw blood from multiple sites through the indwelling IV catheter, without requiring an additional vein puncture.

### SUMMARY OF THE INVENTION

Provided herein is a blood draw device for use with a catheter assembly. The blood draw device includes a housing having a proximal end and a distal end, with the housing defining a housing lumen at the distal end and defining an inner volume that is proximal to the housing lumen. The blood draw device also includes a coupling device positioned at the distal end of the housing and configured to mate with the catheter assembly, a catheter tube movably received within the housing, and an advancement member movably coupled to the housing and configured to move relative to the housing to move the catheter tube between a first position, in which the catheter tube is disposed within the housing, and a second position, in which a distal end of the catheter tube is disposed beyond the distal end of the housing. The blood draw device further includes a first port provided on the housing at the distal end and in fluid communication with the housing lumen, a second port provided proximally from the housing and in fluid communication with the catheter tube, and a fluid flow preventing seal positioned in the housing lumen and proximal to the first port, wherein the distal end of the catheter tube is proximal to the fluid flow preventing seal when in the first position and extends through the fluid flow preventing seal when in the second position.

In some embodiments, the housing lumen includes a main channel extending from the coupling device to the inner volume and a secondary channel extending from the main channel to the first port, the secondary channel joining to the main channel at a T-junction.

In some embodiments, the fluid flow preventing seal is positioned within the main channel proximal to the T-junction.

In some embodiments, the fluid flow preventing seal is a split septum and, when the catheter tube is in the first position, the distal end of the catheter tube is positioned proximal to the split septum, such that split septum is closed and prevents fluid flow to the inner volume and to the catheter tube and the second port, and when the catheter tube is in the second position, the catheter tube extends through the split septum.

In some embodiments, the coupling device is a male luer connector, the male luer connector including an elongated member and a rotating collar, the rotating collar configured to engage a connector of the catheter assembly in a threading fashion.

In some embodiments, the coupling device is a lock including a blunted cannula and a pair of locking arms for coupling to a connector of the catheter assembly.

In some embodiments, the advancement member is a telescopic cylinder having a proximal end and a distal end, with the distal end of the telescopic cylinder positioned within the inner volume and engaged with a proximal end of the catheter tube, and with the telescopic cylinder configured to move relative to the housing to move the catheter tube between the first position and the second position.

In some embodiments, the second port is connected to the proximal end of the telescopic cylinder, and the telescopic cylinder includes a fluid path formed therethrough that fluidly connects the catheter tube to the second port.

In some embodiments, the advancement member includes a first portion movably disposed along an upper surface of the housing and a second portion is movably disposed within the inner volume, with the advancement member seated within a slot formed in the upper surface of the housing and movable therethrough, and wherein a proximal end of the catheter tube is connected to the second portion.

In some embodiments, the blood draw device includes a secondary catheter having a proximal end and a distal end, with the distal end of the secondary catheter coupled to the second portion and extending out proximally therefrom and out the proximal end of the housing, wherein the second port is connected to the proximal end of the secondary catheter and is fluidly connected to the catheter tube via the secondary catheter.

In some embodiments, each of the first port and the second port is a vacutainer port including an outer collar and a needle positioned within a circumference of the outer collar and secured to the housing, with the needle further including a needle lumen therein in fluid communication with the housing lumen and a needle tip positioned to pierce a seal of a vacutainer connected to the vacutainer port, thereby placing an inner chamber of the vacutainer in fluid communication with the needle lumen.

In some embodiments, each of the first port and the second port is a female luer connector configured to couple with a vacutainer via an intervening luer lock access device.

In some embodiments, each of the first port and the second port further includes a needle-free connector attached to the female luer connector, the needle-free connector including a septum therein configured to seal off the female luer connector, and wherein the needle-free connector is configured to couple with a vacutainer via an intervening luer lock access device.

In some embodiments, the needle-free connector is integrated with the female luer connector or solvent bonded to the female luer connector.

Also provided herein is a method for acquiring a pair of blood samples includes connecting a blood draw device to a catheter assembly including an indwelling catheter, with the blood draw device including a housing having a proximal end and a distal end, the housing defining a housing lumen at the distal end and defining an inner volume that is proximal to the housing lumen, a catheter tube movably received within the housing, an advancement member movably coupled to the housing and configured to move relative to the housing to move the catheter tube between a first position, in which the catheter tube is disposed within the housing, and a second position, in which a distal end of the catheter tube is disposed beyond the distal end of the housing and beyond a distal end of the indwelling catheter, a first port provided on the housing at the distal end and in fluid communication with the housing lumen, a second port provided proximally from the housing and n fluid communication with the catheter tube, and a fluid flow preventing seal positioned in the housing lumen and proximal to the first port, wherein the distal end of the catheter tube is proximal to the fluid flow preventing seal when in the first position and extends through the fluid flow preventing seal when in the second position. The method also includes connecting a first vacutainer to the first port with the catheter tube in the first position, with a first blood sample being drawn into the first vacutainer upon connection thereof to the first port, and wherein the fluid flow preventing seal is closed with the catheter tube in in the first position to prevent a flow of blood into the catheter tube and to second port. The method further includes actuating the advancement member to move the catheter tube to the second position and connecting a second vacutainer to the first port with the catheter tube in the second position, with a second blood sample being drawn into the second vacutainer upon connection thereof to the second port. The first blood sample is acquired from a first location in a patient vasculature adjacent the distal end of the indwelling catheter and the second first blood sample is acquired from a second location in the patient vasculature adjacent the distal end of the catheter tube.

In some embodiments, connecting the blood draw device to the catheter assembly includes engaging a rotatable collar of a coupling device of the blood draw device to a connector of the catheter assembly.

In some embodiments, actuating the advancement member includes actuating a telescopic cylinder distally within the housing to move the catheter tube to the second position, the second port connected to a proximal end of the telescopic cylinder.

In some embodiments, actuating the advancement member comprises distally actuating a first portion, in the form of a tab, along an upper surface of the housing, with the first portion joined to a second portion movably disposed within the inner volume, and wherein actuation of the first member causes the advancement member to move within a slot formed in the upper surface of the housing.

In some embodiments, connecting each of the first vacutainer to the first port and the second vacutainer to the second port includes connecting a respective vacutainer to a vacutainer port having an outer collar and a needle positioned within a circumference of the outer collar, the needle including a needle lumen. In connecting the respective vacutainer to the vacutainer port, the needle pierces a seal of the vacutainer to place an inner chamber of the vacutainer in fluid communication with the needle lumen and further in fluid communication with the housing lumen.

In some embodiments, connecting each of the first vacutainer to the first port and the second vacutainer to the second port includes connecting a luer lock access device to a female luer connection of the respective port or a female luer connector of a needle-free connector attached to the respective port, the luer lock access device having a male luer connection and a needle including a needle lumen, and also connecting a respective vacutainer to the luer lock access device. In connecting the respective vacutainer to the luer lock access device, the needle pierces a seal of the vacutainer to place an inner chamber of the vacutainer in fluid communication with the needle lumen and further in fluid communication with the housing lumen.

### BRIEF DESCRIPTION OF THE DRAWINGS

FIG. 1 is a side view of a system including a catheter assembly and an associated blood draw device useable therewith, according to a non-limiting embodiment described herein;

FIG. 2 is a perspective view of the blood draw device included in the system of FIG. 1;

FIG. 3 is a partial cross-section view of the system of FIG. 1, taken along line 3-3, according to an embodiment described herein;

FIG. 4 illustrates the system of FIG. 1, with the blood draw device in a first configuration and a vacutainer attached to a first port;

FIG. 5 illustrates the system of FIG. 1, with the blood draw device in a second configuration and a vacutainer attached to a second port;

FIG. 6 is a side view of a system including a catheter assembly and an associated blood draw device useable therewith, according to another non-limiting embodiment described herein;

FIG. 7 is a side view of a system including a catheter assembly and an associated blood draw device useable therewith, according to another non-limiting embodiment described herein;

FIG. 8 is a side view of a system including a catheter assembly and an associated blood draw device useable therewith, according to a non-limiting embodiment described herein;

FIG. 9 is a cross-section view of the blood draw device included in the system of FIG. 8, taken along line 3-3, according to an embodiment described herein;

FIG. 10 illustrates the system of FIG. 8, with the blood draw device in a first configuration and a vacutainer attached to a first port; and

FIG. 11 illustrates the system of FIG. 8, with the blood draw device in a second configuration and a vacutainer attached to a second port.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device being manipulated by the user would be the proximal end of the device.

The terms “first”, “second”, and the like are not intended to refer to any particular order or chronology, but refer to different conditions, properties, or elements.

As used herein, “at least one of” is synonymous with “one or more of.” For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

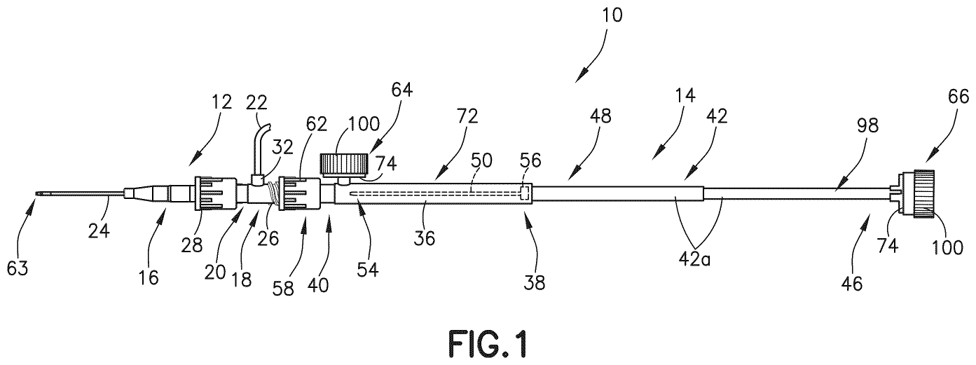

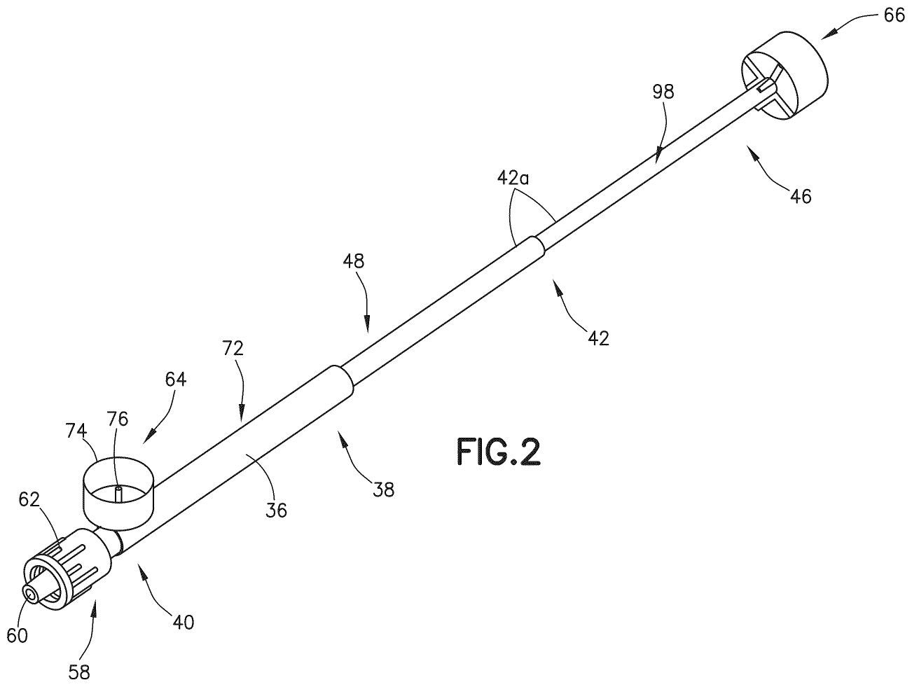

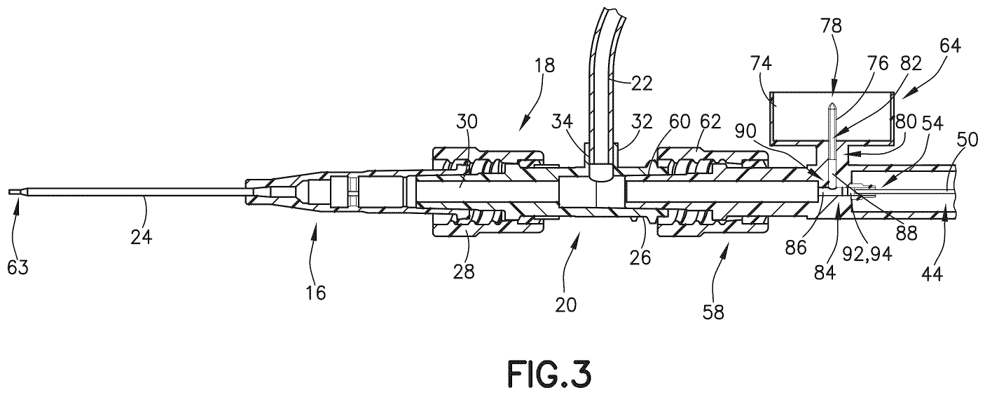

Referring now to FIGS. 1-5, shown is a non-limiting embodiment of a system 10 including a catheter assembly 12 and blood draw device 14. The catheter assembly 12 includes a catheter hub 16 and a non-integrated extension set 18 that includes a catheter connector 20 and (optionally) extension tubing 22. The catheter hub 16 receives a catheter 24 and is coupled to the catheter connector 20. In one aspect or embodiment, the catheter hub 16 is the AccuCath™ catheter system commercially available from Becton, Dickinson and Company. The catheter 24 may be employed as a peripheral intravenous catheter, a midline catheter, or a peripherally-inserted central catheter, and the catheter 24 may be formed of any suitable material and may be of any useful length, as known to those of skill in the art.

The catheter connector 20 is configured to be placed in contact with the skin surface of a patient at or near an insertion site of catheter 24. While not shown, it is recognized that the catheter connector 20 may include stabilization features (e.g., wings) thereon that assist with retaining the catheter connector 20 in place on the skin of a patient. The catheter connector 20 includes a proximal port with a coupler 26 and a distal port with a distal coupler 28, and defines at least one lumen 30 extending through or otherwise in fluid communication with the couplers 26 and 28. The proximal coupler 26 and/or the distal coupler 28 can be, for example, male or female luer locks and/or any other suitable coupler, with the non-limiting embodiment of FIGS. 1-5 illustrating the proximal coupler 26 as a female luer connector and the distal coupler 28 as a male luer connector. The proximal coupler 26 can be physically and fluidically coupled to the blood draw device 14 to enable a multi-site blood draw, as will be explained in further detail below. The distal coupler 28 can be physically and fluidically coupled to catheter hub 16 such that the lumen 30 of the catheter connector 20 is at least selectively in fluid communication with the catheter 24.

The catheter connector 20 may also include and/or define one or more additional ports, such as a side port 32. In some embodiments, side port 32 may be distal to the proximal coupler 26 and proximal to the distal coupler 28. Positioning side port 32 in such a position along a length of the catheter connector 20, between the couplers 26 and 28, can facilitate flushing and/or fluid transfer via side port 32. Side port 32 defines a lumen 34 that is in fluid communication with the lumen 30 between distal coupler 28 and proximal coupler 26. In other words, the catheter connector 20 can include and/or define a first lumen 30 between distal coupler 28 and proximal coupler 26 and a second lumen 34 defined by side port 32. As such, side port 32 can provide access to the lumen 30 between distal coupler 28 and proximal coupler 26, which in turn can provide access to the catheter hub 12 (and catheter 24) that is coupled to the distal coupler 28.

In some embodiments, the arrangement of side port 32 can be such that the catheter connector 20 forms, for example, a Y-connector or a T-connector. More particularly, side port 32 can be disposed substantially perpendicular (e.g., about 90 degrees) to the lumen 30 of the catheter connector 20 and near or adjacent the proximal coupler 26. In non-limiting embodiments, side port 32 extends from catheter connector 20 at an angle, relative to the lumen 30 between distal coupler 28 and proximal coupler 26, that is not 90 degrees (e.g., side port 32 extends at an angle of, for example and without limitation, 15-165 degrees, with all values and subranges therebetween inclusive).

As previously indicated, it is recognized that extension set 18 may include extension tubing 22 that is connected to side port 32 and in fluid communication with the lumen 34 of the side port 32. In some embodiments, the side port 32 and/or the extension tubing 22 can be and/or can form at least a portion of a fluid line that can be used to deliver fluid, remove fluid, flush fluid, and/or the like. In such embodiments, for example, an arrangement in which side port 32 is disposed adjacent to the proximal coupler 26 can enable flushing of the proximal coupler 26, any valve(s) included therein, and/or a space between any valve(s) and an inner surface of the catheter connector 20.

With continuing reference to FIGS. 1-5 blood draw device 14 is shown as including a housing 36 having a proximal end 38 and a distal end 40, and an advancement member 42 slideably received within housing 36 (i.e., within an inner volume 44 of housing 36). In the illustrated embodiment, the advancement member 42 is provided as one or more telescopic cylinders 42a that are provided in a telescoping relationship with housing 36, such that advancement member 42 may be slideably received entirely, or almost entirely, within the inner volume 44 of housing 36. Advancement member 42 also includes a proximal end 46 and a distal end 48 and, in non-limiting embodiments, advancement member 42 may have a variable diameter along its length. As one example, the distal end 48 of advancement member 42 may have a larger diameter than other portions of advancement member 42 such that, as advancement member 42 is retracted, one or more features on housing 36 may interact with the enlarged portion of advancement member 42 to prevent pulling advancement member 42 completely out of housing 36. As another example, the distal end 48 of advancement member 42 may have a smaller diameter than other portions of advancement member 42, to keep the advancement member 42 in position at a blood draw forward condition, so that a hand of the operator is freed up to manipulate additional components (e.g., a vacutainer tube).

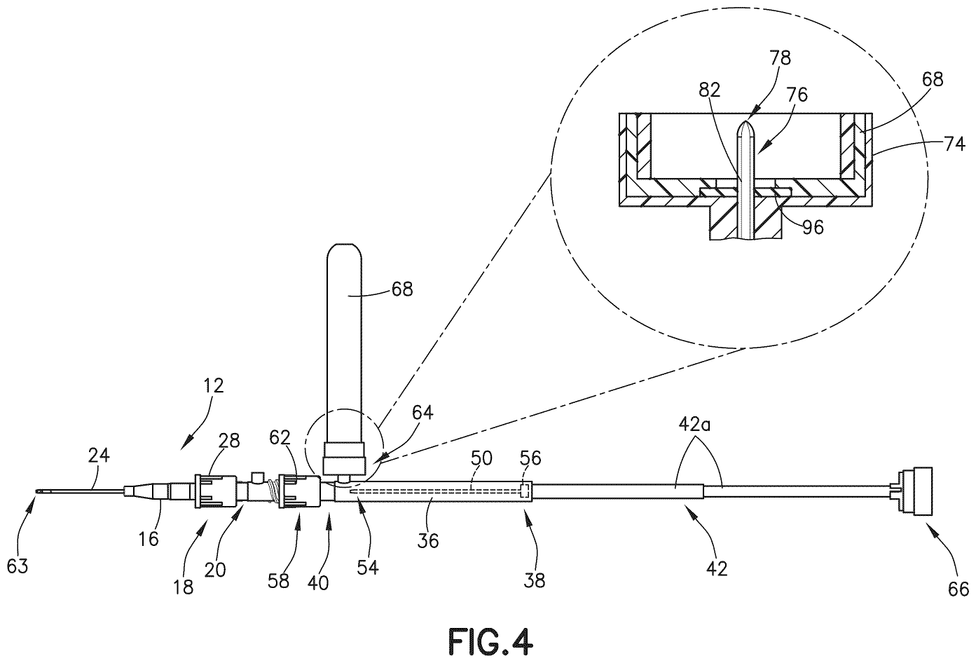

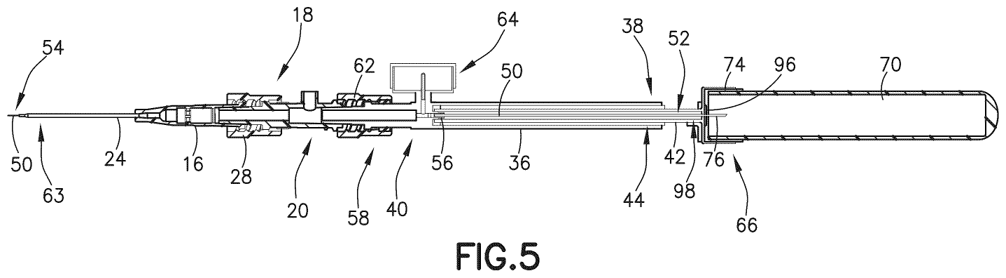

Blood draw device 14 further includes a catheter tube 50 having a proximal end 52 and a distal end 54. Catheter tube 50 is received within the inner volume 44 of housing 36, and may be advanced and/or retracted relative to housing 36 by displacement of the advancement member 42 relative to the housing 36. In some embodiments, the catheter tube 50 may be joined to advancement member 42 via a fitting 56 provided at the distal end 48 of advancement member 42, such that displacement of the advancement member 42 relative to the housing 36 causes a corresponding displacement of catheter tube 50. In non-limiting embodiments, catheter tube 50 may be advanced from a first positon as shown in FIG. 4, in which distal end 54 of catheter tube 50 is within housing 36, to a second position as shown in FIG. 5, in which a distal end 54 of catheter tube 50 is positioned distally of housing 36 and also positioned distally of catheter 24, as will be explained in further detail below.

As shown in FIGS. 1-5, a coupling device 58 is provided at the distal end 40 of housing 36 by which blood draw device 14 may be coupled to catheter assembly 12, i.e., to connector 20. The coupling device 58 is exemplified as a male luer connector that mates with the female luer connector of proximal coupler 26 on connector 20, with the male luer connector of coupling device 58 having an elongated member 60 surrounded by a rotating collar 62. The rotating collar 62 may be rotated to threadingly engage the male luer connector to the female luer connector of proximal coupler 26. However, it is appreciated that alternative embodiments of blood draw device 14 may include a coupling device 58 of another type to secure blood draw device 14 to catheter assembly 12, including luer connections, clips, blunt plastic cannulae, blunt metal cannulae, hybrid luers (e.g., with a cannula) friction fits, and the like.

As can be appreciated, FIG. 4 shows blood draw device 14 in a first state, where catheter tube 50 is in a first position, received within housing 36, and advancement member 42 is in a first position, extending proximally from housing 36. The advancement member 42 may then be advanced distally relative to the housing 36 by way of a user applying a distally-directed force to advancement member 42. As indicated above, advancement member 42 is coupled to catheter tube 50 (e.g., by fitting 56) and/or otherwise interacts catheter tube 50 such that, as advancement member 42 is advanced distally to a second position, as shown in FIG. 5, catheter tube 50 is moved to a second position, where a distal end 54 of catheter tube 50 extends beyond housing 36 and may also extend past the distal tip 63 of catheter 24.

According to embodiments, the blood draw device 14 includes two ports 64, 66 thereon that provide for connection of multiple blood collection devices to the blood draw device 14, so as to enable collection of two separate blood samples (i.e., a paired blood draw) via the device 14. In one embodiment, and as illustrated in FIGS. 1-5, the ports 64, 66 are configured as vacutainer ports configured to receive therein vacutainers 68, 70 used for collection of the blood samples—with a first vacutainer port 64 provided to receive a first vacutainer 68 and a second vacutainer port 66 provided to receive a second vacutainer 70.

The first vacutainer port 64 is provided on housing 36 adjacent the distal end 40 thereof. In accordance with one embodiment, the first vacutainer port 64 may be provided on an upper surface 72 of the housing 36 to provide for easy connection of the first vacutainer 68 thereto. The first vacutainer port 64 includes an outer collar 74 that is positioned on the upper surface 72 of housing 36 and extends outwardly therefrom. In some embodiments, an inner surface of the outer collar 74 may be configured as a threaded inner surface. The first vacutainer port 64 also includes a hollow needle or cannula 76 that is positioned partially within the circumference of outer collar 74. The needle 76 is positioned such that a tip 78 of needle 76 extends approximately up to an upper edge of the outer collar 74, with the needle 76 extending downwardly therefrom through a bottom opening 80 of outer collar 74 and being secured to housing 36. Within the housing 36, a lumen 82 (hereafter “needle lumen 82”) of needle 76 is joined with a lumen 84 (hereafter “housing lumen 84”) defined within the distal end 40 of housing 36. The housing lumen 84 includes a main channel 86 and a secondary channel 88, with the main channel 86 extending lengthwise between the coupling device 58 (and through coupling device 58, so as to be in fluid communication with the lumen 30 in catheter connector 20 when blood draw device 14 is coupled to catheter connector 20) and the inner volume 44, and the secondary channel 88 oriented orthogonally to the main channel 86 and joining therewith at a T-junction 90. The needle lumen 82 is joined with the secondary channel 88 of housing lumen 84.

According to embodiments of the disclosure, a fluid flow preventing seal 92 may be provided in the housing lumen 84 at a location proximal from the T-junction 90 that may prevent a flow of fluid (i.e., blood) from the housing lumen 84 into the inner volume 44 of housing 36. In some embodiments, the fluid flow preventing seal 92 is formed as a split septum (hereafter “split septum 92”) that is positioned within housing lumen 84 just proximal to the T-junction 90. The split septum 92 may be formed of a flexible, elastomeric material according to one embodiment, and includes a slit 94 formed therein through which the catheter tube 50 may pass when it is advanced from the first position to the second position. In some embodiments, the slit 94 is configured to stretch when the catheter tube 50 is passed therethrough. Based on the sizing of the slit 94, a seal is formed between the septum 92 and the catheter tube 50 as the catheter tube 50 is advanced through the slit 94 when the catheter tube 50 is moved to the second position.

In attaching a first vacutainer 68 to the first vacutainer port 64, a front portion of the first vacutainer 68—which may include a pierceable seal or cover 96 thereon—is positioned within the outer collar 74 (and, in some embodiments, is engaged in a threading manner with the outer collar 74) and is forced down onto needle 76, such that tip 78 of needle 76 pierces the seal/cover 96. Upon insertion of the needle 76 into first vacutainer 68, the needle lumen 82 is placed in fluid communication with an interior volume of the first vacutainer 68, with the vacutainer 68 thus also in fluid communication with housing lumen 84 and, by extension, in fluid communication with the catheter 24 (via catheter connector 20). Accordingly, a blood sample may be collected in first vacutainer 68 upon connection thereof to first vacutainer port 64 of blood draw device 14.

The second vacutainer port 66 is provided on blood draw device 14 proximally from housing 36. According to one embodiment, the second vacutainer port 66 is positioned at and connected to the proximal end 46 of advancement member 42, to allow for connection of the second vacutainer 70 thereto. According to another embodiment, the second vacutainer port 66 is positioned at and connected to a proximal end of a separate fluid tube (not shown) that passes through advancement member 42 and extends out proximally therefrom. Similar to the first vacutainer port 64, second vacutainer port 66 includes an outer collar 74 and a hollow needle 76 that is positioned partially within the circumference of outer collar 74. The needle 76 is positioned such that a tip 78 of needle 76 extends approximately up to an upper edge of the outer collar 74, with the needle 76 extending downwardly therefrom to a bottom opening 80 of outer collar 74 and being retained in opening 80. A needle lumen 82 is joined with a lumen or fluid conduit 98 defined within the advancement member 42 (or the separate fluid tube), such that the needle lumen 82 is in fluid communication with the fluid conduit 98 in advancement member 42.

In attaching a second vacutainer 70 to the second vacutainer port 66, a front portion of the second vacutainer 70—which may include a pierceable seal or cover 96 thereon—is positioned within the outer collar 74 and is pressed down onto needle 76, such that tip 78 of needle 76 pierces the seal/cover 96. Upon insertion of the needle 76 into second vacutainer 70, the needle lumen 82 is placed in fluid communication with an interior volume of the second vacutainer 70, with the vacutainer thus also in fluid communication with fluid conduit 98 of advancement member 42 and, by extension, in fluid communication with the catheter tube 50. Accordingly, a blood sample may be collected in second vacutainer 70 upon connection thereof to second vacutainer port 66 of blood draw device 14.

For each of the first vacutainer port 64 and second vacutainer port 66, components may be provided that serve to shield and protect the ports when not in use. According to some embodiments, a cap 100 (FIG. 1) may be attached to the outer collar 74 of each port 64, 66 to provide protection thereto and to maintain cleanliness of the port 64, 66 and the needle 76 thereof. Additionally, a rubber sleeve (not shown) may be positioned over the needle 76 of each port 64, 66 that maintains cleanliness of the needle 76 and that protects against accidental needle sticks when the blood draw device 14 is being handled by a clinician.

With reference now to FIGS. 4 and 5, operation of blood draw device 14 to obtain first and second blood draws is shown and described herein in further detail. In an initial step, the blood draw device 14 is connected to a catheter assembly 12 that includes therein a catheter 24 that has previously been placed within the vasculature of a patient, so as to provide an indwelling catheter. As previously described, in one embodiment, the blood draw device 14 is connected to catheter assembly 12 via engagement of a male luer connection of coupling device 58 provided at the distal end 40 of housing 36 with a female luer connection of proximal coupler 26 of catheter connector 20. Upon connection of coupling device 58 of housing 36 with proximal coupler 26 of catheter connector 20, the housing lumen 84 at distal end 40 is in fluid connection with the lumen 30 within catheter connector 20.

Upon connection of blood draw device 14 to catheter assembly 12, the first vacutainer 68 is connected to the first vacutainer port 64, with a front portion of the first vacutainer 68 positioned within the outer collar 74 and pressed down onto needle 76, such that the tip 78 of needle 76 pierces a seal/cover 96 of the first vacutainer 68, as shown in FIG. 4. Upon connection of the first vacutainer 68, the vacuum within first vacutainer 68 causes a flow of blood to commence through catheter 24, through catheter hub 14, through the lumen 30 of catheter connector 20, through a portion of the housing lumen 84 (i.e., through distal part of main channel 86 and through secondary channel 88), and through the needle lumen 82 and into the first vacutainer 68—such that a first blood sample is collected therein. The first blood sample collected by first vacutainer 68 is obtained from a location within the vasculature of the patient where the distal tip 63 of indwelling catheter 24 is positioned. It is noted that, during collection of the first blood sample, the split septum 92 in blood collection device (which is positioned in main channel 86 proximal from the T-junction 90) prevents blood from flowing proximally into the inner volume 44 of housing 36.

Upon collection of the first blood sample, the first vacutainer 68 may be disconnected from the first vacutainer port 64 and the blood draw device 14 may then be operated to acquire a second blood sample. As illustrated in FIG. 5, in preparing to acquire the second blood sample, the advancement member 42 of blood draw device 14 may be actuated (i.e., moved distally) to cause the catheter tube 50 to move from the first position, in which distal end 54 of catheter tube 50 is within housing 36 and proximal to the split septum 92, to the second position, in which a distal end 54 of catheter tube 50 is advanced distally through split septum 92 and out from housing 36 and further out distally past the distal end 63 of indwelling catheter 24, such as by a distance of 10 mm, as a non-limiting example.

Upon advancing of the catheter tube 50 to a desired position (i.e., with distal tip 54 of the catheter tube 50 extended distally past the distal tip 63 of indwelling catheter 24), the second vacutainer 70 is connected to the second vacutainer port 66, with a front portion of the second vacutainer 70 positioned within the outer collar 74 and pressed down onto needle 76, such that the tip 78 of needle 76 pierces a seal/cover 96 of the second vacutainer 70. Upon connection of the second vacutainer 70, the vacuum within second vacutainer 70 causes a flow of blood to commence through catheter tube 50, through fluid conduit 98 of advancement member 42, and through the needle lumen 82 and into the second vacutainer 70—such that a second blood sample is collected therein. The second blood sample collected by second vacutainer 70 is obtained from a location within the vasculature of the patient where the distal tip 54 of catheter tube 50 is positioned, which is different from the location at which the first blood sample was previously collected (at the tip 63 of indwelling catheter 24). It is noted that, during collection of the second blood sample, the advancement/positioning of the catheter tube 50 through split septum 92 and housing lumen 84 prevents blood from flowing proximally into the inner volume 44 of housing 36 and also prevents blood from flowing into/out the first vacutainer port 64.

Accordingly, blood draw device 14 provides for a blood draw to be performed from multiple sites through the indwelling catheter without additional vein puncture. Two separate blood samples may be obtained at two separate sites within the patient vasculature—with a first sample collected through indwelling catheter 24 at a location of the distal tip 63 thereof and provided to a first vacutainer 68 coupled to blood draw device 14 and a second sample collected through the advanced catheter tube 50 at a location of the distal tip 54 thereof and provided to a second vacutainer 70 coupled to blood draw device 14. The two separate blood samples drawn from different locations may be used, for example, as a paired blood culture set (PBC) that may be tested for bacteremia and/or other contaminants, with the PBC providing a high microbiologic yield and an increased/improved sensitivity to detect pathogens and time to positivity (TTP), as compared to a single blood culture (SBC).

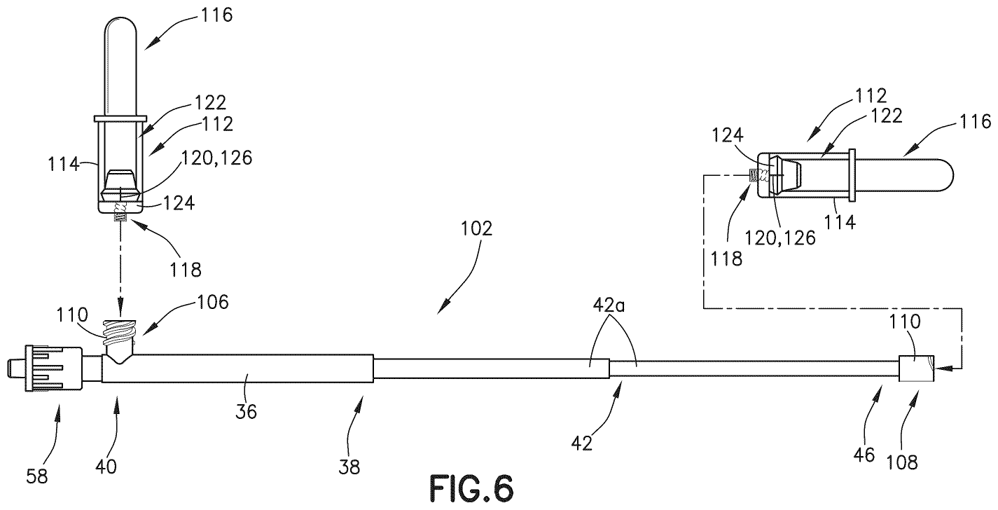

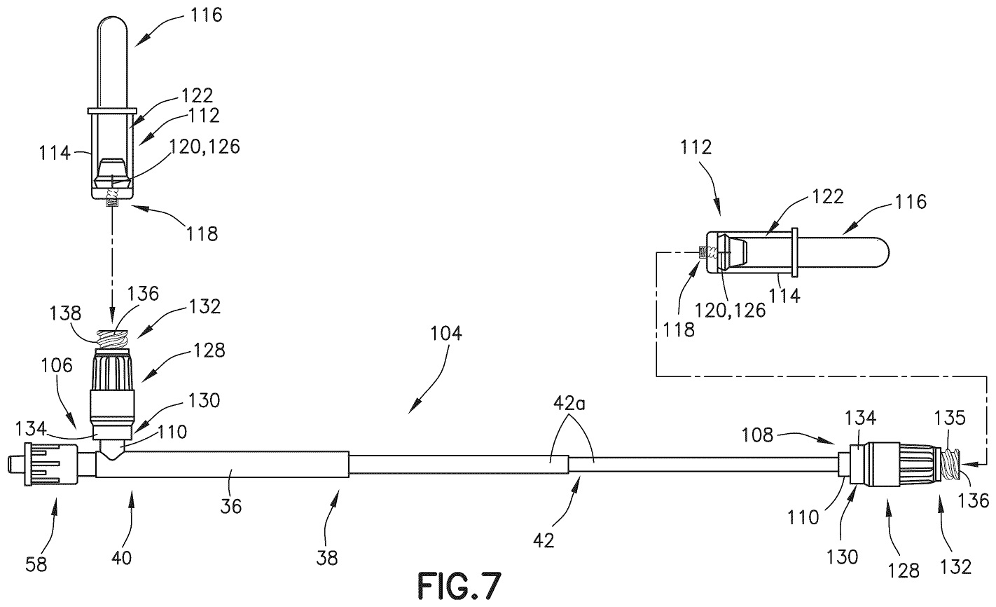

Referring now to FIG. 6 and FIG. 7, blood draw devices 102, 104 are shown according to additional embodiments of the disclosure. The blood draw devices 102, 104 are identical in design and operation as the blood draw device 14 of FIGS. 1-5 except that the structure of the first and second ports provided thereon differ from the ports of blood draw device 14, such that the structure of vacutainers (and an accompanying access member) connected thereto is also modified to match such structure. The positioning of the ports on housing 36 and advancement member 42, and the fluid connection of the ports to the housing lumen 84 and the fluid conduit 98 in advancement member 42, are as previously described, and thus are not repeated hereafter.

Referring first to FIG. 6, a blood draw device 102 is provided that includes a first port 106 and a second port 108 structured as luer connections. Specifically, each of ports 106, 108 is configured as a female luer connection 110 that may include a tapered cavity (not shown) configured to receive and engage a tapered stem or elongated member of a male luer connection, as well as a threaded outer surface configured to engage threads on the inner surface of an annular shield of a male luer connection. As previously described, the first port 106 is positioned on housing 36 adjacent the distal end 40 thereof and may be provided on an upper surface 72 of the housing 36, while the second port 108 is positioned at the proximal end 46 of advancement member 42.

According to aspects of the disclosure, an access device 112 such as a BD Vacutainer® Luer-Lok™ access device from Becton Dickinson and Company, for example, is coupleable to the female luer connection 110 of each of the first port 106 and the second port 108. The access device 112 generally includes an outer sleeve 114 configured to receive a vacutainer 116, as well as a male luer connection 118 extending out from the outer sleeve 114 and a needle 120 physically and fluidly connected to the male luer connection 118 that extends into a cavity 122 defined by the outer sleeve 114. The male luer connection 118 of access device 112 may be coupled to the female luer connection 110 of the respective first or second port 106, 108, to secure the access device 112 to the blood draw device 102 and to fluidly connect the needle 120 thereof to the female luer connection 110. Upon connection of the access device 112 to the respective first or second port 106, 108, a vacutainer 116 may be inserted into the outer sleeve 114 of the access device 112 and forced down onto the needle 120 thereof, such that a tip of needle 120 pierces a seal/cover 124 of the vacutainer 116. Upon insertion of the needle 120 into the vacutainer 116, a lumen 126 of needle 120 is placed in fluid communication with an interior volume of the vacutainer 116, with the vacutainer 116 thus also in fluid communication with the blood draw device 102. Specifically, a first vacutainer 116 connected to first port 106 is placed in fluid communication with housing lumen 84 at the distal end 40 of housing 36 (and, by extension, in fluid communication with the catheter 24, via catheter connector 20 and catheter hub 12), while the second vacutainer 116 connected to second port 108 is placed in fluid communication with the fluid conduit 98 of advancement member 42 (and, by extension, in fluid communication with the catheter tube 50). Accordingly, a blood sample may be collected in the first and second vacutainers 116 upon connection thereof to the respective first and second ports 106, 108 of blood draw device 102.

Referring now to FIG. 7, a blood draw device 104 is provided that includes a first port 106 and a second port 108 structured as female luer connections 110 with a needle-free connector (NFC) 128 provided thereon. According to embodiments, the NFCs 128 may be integrated with the female luer connections 110 or bonded thereto, with the integrated NFCs 128 being permanently connected to the luer connections 110 and the bonded NFCs 128 being solvent bonded to the luer connections 110 via a suitable solvent (such as acetone, as a non-limiting example). The NFCs 128 integrated/bonded with the female luer connections 110 seal off the respective ports 106, 108 on the blood draw device 104. According to embodiments, the NFC 128 on each of first port 106 and second port 108 may be a known NFC 128, including without limitation, a BD Q-Syte™, BD MaxZero™, BD MaxPlus™, or SmartSite™ needle free connector by Becton Dickinson and Company.

In general, the NFC 128 may include a male luer connection 130 and a female luer connection 132. The male luer connection 130 of the NFC 128 is connected to the female luer connection 110 formed on housing 36 and may include a tapered stem (not shown) received by a corresponding tapered cavity of the female luer connection 110, as well as an annular shield 134 extending about the tapered stem that includes threads on an inner surface thereof configured to engage corresponding threads on an outer surface of the female luer connection 110. The female luer connection 132 of NFC 128 includes an elongated proximal end portion with a cover or septum 136 (including a slit) positioned over an opening of a tapered cavity configured to receive and engage a corresponding tapered stem or elongated member of a male luer connection (of an access device 112, as described below). The female luer connection 132 can also include an outer surface that includes threads 138 configured to engage corresponding threads on an inner surface of an annular shield of a male luer connection.

Similar to the blood draw device of FIG. 6, an access device 12 (e.g., BD Vacutainer® Luer-Lok™ access device) may be coupled to the NFC 128 of each of the first port 106 and the second port 108. As previously described, the access device 112 generally includes an outer sleeve 114 configured to receive a vacutainer 116, as well as a male luer connection 118 extending out from the outer sleeve 114 and a needle 120 physically and fluidly connected to the male luer connection 118 that extends into a cavity 122 defined by the outer sleeve 114. The male luer connection 118 of access device 112 may be coupled to the female luer connection 132 of the NFC 128 provided at the respective first or second port 106, 108, to secure the access device 112 thereto and to fluidly connect the needle 120 thereof to the blood draw device 104. Upon connection of the access device 112 to the respective first or second port 106, 108, a vacutainer 116 may be inserted into the outer sleeve 114 of the access device 112 and forced down onto the needle 120 thereof, such that a tip of needle 120 pierces a seal/cover of the vacutainer 116. Upon insertion of the needle 120 into the vacutainer 116, a lumen 126 of needle 120 is placed in fluid communication with an interior volume of the vacutainer 116, with the vacutainer 116 thus also in fluid communication with the blood draw device 104. Specifically, a first vacutainer 116 connected to first port 106 is placed in fluid communication with housing lumen 84 (and, by extension, in fluid communication with the catheter 24, via catheter connector 20 and catheter hub 12), while the second vacutainer 116 connected to second port 108 is placed in fluid communication with the fluid conduit 98 of advancement member 42 (and, by extension, in fluid communication with the catheter tube 50). Accordingly, a blood sample may be collected in the first and second vacutainers 116 upon connection thereof to the respective first and second ports 106, 108 of blood draw device 104.

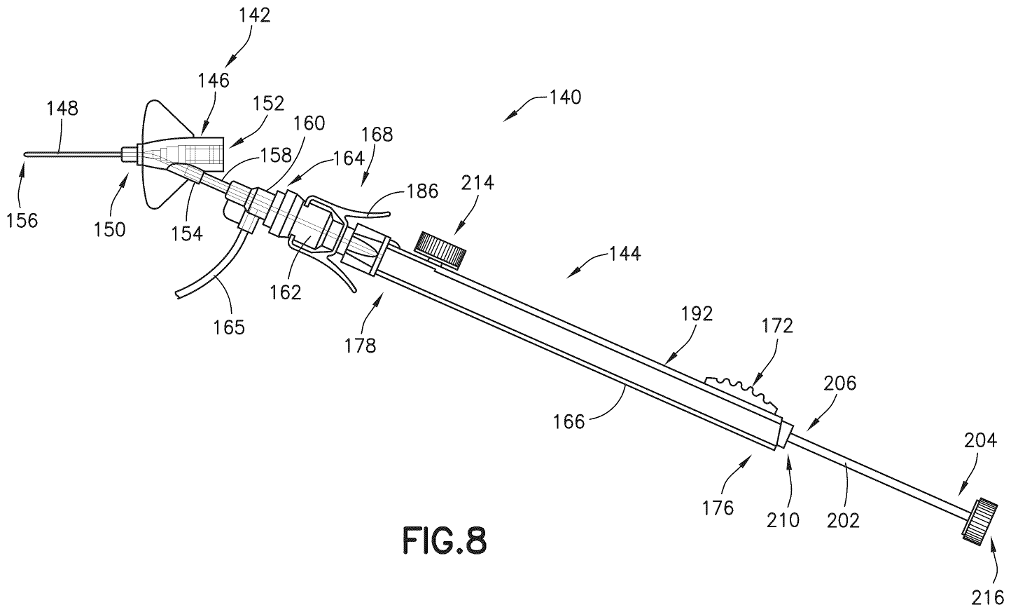

Referring now to FIGS. 8-11, shown is another non-limiting embodiment of a system 140 including a catheter assembly 142 and blood draw device 144. The catheter assembly 142 may include a catheter adapter 146 and associated catheter 148. The catheter adapter 146 includes a distal end 150 and a proximal end 152 and may include an additional adapter port 154 that may be disposed therebetween. The catheter 148 extends from the distal end 150 of catheter adapter 146, with a distal end or tip 156 of the catheter 148 positioned appropriately within the vasculature of a patient to enable a blood draw.

In some non-limiting embodiments or aspects, the catheter assembly 142 may include a fluid conduit 158 extending from the port 154 and out to a connector 160. Connector 160 may be a t-connector (e.g., one side port arranged at a 90 degree angle relative to a longitudinal axis of connector 160), a y-connector (e.g., one side port arranged at a 25, a 60, or a 75 degree angle relative to a longitudinal axis of connector 160), or any other type of connector known in the art. In some non-limiting embodiments or aspects, connector 160 may have a needleless access connector 162 coupled to the proximal end 164 thereof, with the needleless access connector 162 providing a near-patient access port to the catheter assembly 142. In some non-limiting embodiments or aspects, catheter assembly 142 may also include an extension tube/extension set 165 coupled to the port 154 of the connector 160 that may be used to provide fluid from a fluid source (e.g., IV bar), for example.

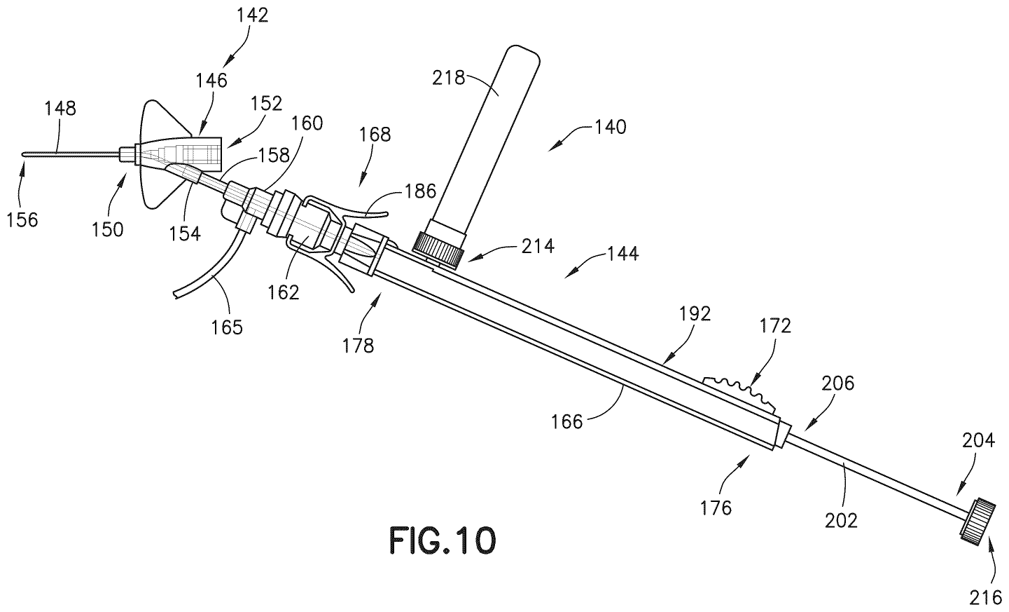

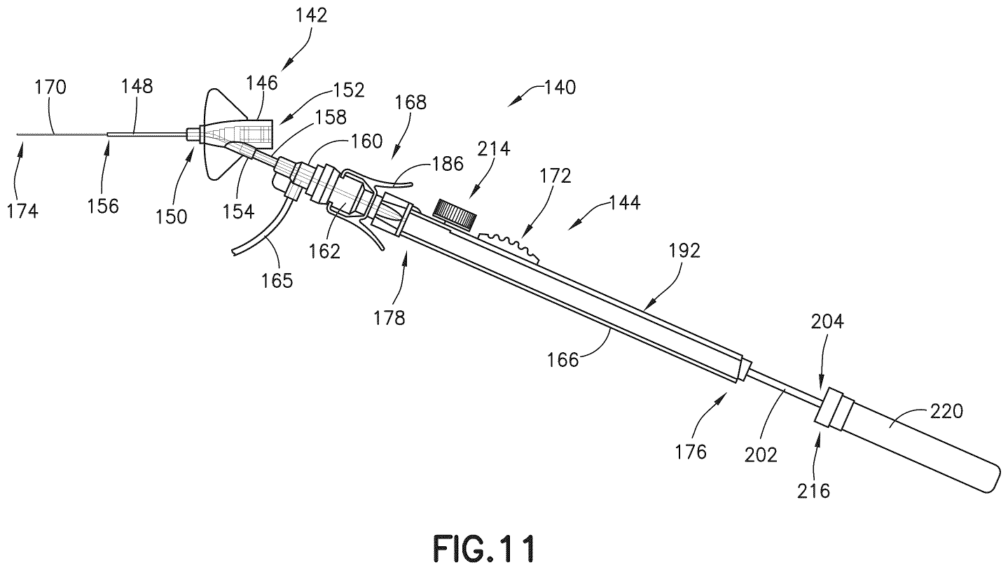

As shown in FIGS. 8-11, according to a non-limiting embodiment, the blood draw device 144 includes at least a housing 166, a coupling device 168, a catheter tube 170, and an advancement member 172. As will be described in further detail below, the catheter tube 170 is moveable within the housing 166 so as to provide for advancement of a portion of the catheter tube 170 from a first or retracted position inside the housing 166 (FIG. 10) to a second or advanced position outside of the housing 166 (FIG. 11), such that a distal end 174 of the catheter tube 170 may be routed into the catheter assembly 142 and out past a distal tip 156 of catheter 148.

As shown in FIGS. 8-11, the housing 166 of blood draw device 144 can be an elongate member having a proximal end 176 and a distal end 178 and defining an inner volume 180. The housing 166 may include one or more features or surface finishes on an outer surface thereof that can be arranged to increase the ergonomics of the blood draw device 144, which in some instances can allow a user to manipulate the blood draw device 144 with one hand (i.e., single-handed use).

The coupling device 168 of blood draw device 144 is provided at the distal end 178 of the housing 166, with the coupling device 168 providing for reversible coupling of the blood draw device 144 to catheter assembly 142, such as via needleless access connector 162. In some embodiments, the coupling device 168 is configured as a lock 182 that includes a blunted cannula 184 and locking arms 186 for coupling to the needleless access connector 162 of catheter assembly 142, with the blunted cannula 184 and locking arms 186 forming three points of contact therewith. However, those of skill will appreciate that any connection or coupling, for example a luer, can be used, so long as the distal end 174 of catheter tube 170 may pass through the coupling device 168 to catheter assembly 142.

The advancement member 172 of blood draw device 144 includes a first portion 188 and a second portion 190. The first portion 188 is movably disposed along an upper surface 192 of the housing 166 and the second portion 190 is movably disposed within the inner volume 180 of the housing 166. The arrangement of the advancement member 172 and the housing 166 is such that a connecting portion (not shown) of the advancement member 172 that joins the first and second portions 188, 190 is seated within a slot 194 formed in the upper surface 192 of the housing 166—the slot 194 generally extending a majority of the way between the proximal and distal ends 176, 178 of the housing 166. As the first and second portions 86, 88 are joined together, movement of the first portion 188 along the upper surface 192 of the housing 166 results in a corresponding movement of the second portion 190 within the inner volume 180.

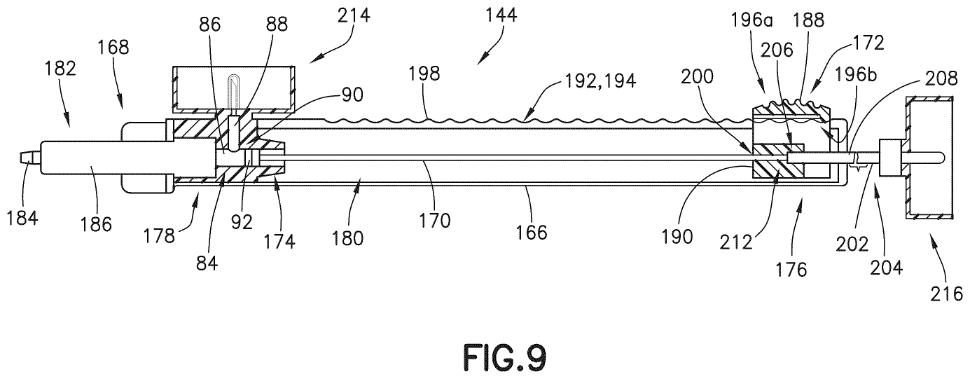

As shown best in FIG. 9, the first portion 188 of the advancement member 172 may be configured as a tab or tab having a contact surface 196a engageable by a user and an underside 196b that is in contact with the upper surface 192 of the housing 166. In such embodiments, the upper surface 192 of the housing 166 can include a track 198, for example, a set of ribs, ridges, bumps, grooves, and/or the like along which the underside 196b of tab or protrusion advances when the advancement member 172 is engaged by a user. In this manner, a user can engage the first portion 188 of the advancement member 172 and can move the advancement member 172 relative to the housing 166.

The second portion 190 includes an opening 200 extending therethrough that is configured to grip or retain a portion of the catheter tube 170. Due to a portion of the catheter tube 170 being retained within the opening 200 of second portion 190, movement of the advancement member 172 relative to housing 166 causes a corresponding movement of the catheter tube 170 relative to the housing 166. In this manner, the distal end 174 of the catheter tube 170 can be selectively moved out of or back into the inner volume 180 of the housing 166 as desired, such as advancing the distal end 174 of the catheter tube 170 out of the housing 166 when the blood draw device 144 has been coupled to the catheter assembly 142 and collection of a blood sample is to be performed.

As further shown in FIGS. 8-11, blood draw device 144 includes a secondary catheter 202 provided at the proximal end 176 of the housing 166. The secondary catheter 202 has a proximal end portion 204 and a distal end portion 206 and defines a lumen 208. A portion of the secondary catheter 202 is disposed within and extends through an opening 210 formed in the proximal end 176 of housing 166. As such, the proximal end portion 204 of secondary catheter 202 is at least partially disposed outside of the housing 166 and the distal end portion 206 is at least partially disposed within the housing 166, with the distal end portion 206 coupled to the second portion 190 of the advancement member 172. In some embodiments, the secondary catheter 202 can have a larger diameter than the catheter tube 170 such that a proximal end 212 of the catheter tube 170 is at least partially disposed within the lumen 208 defined by the secondary catheter 202 when the catheter 66 and the secondary catheter 202 are coupled to the second portion 190 of the advancement member 172.

As with the blood draw devices 14, 102, 104 of FIGS. 1-7, the blood draw device 144 includes two ports 214, 216 thereon that provide for connection of multiple blood collection devices to the blood draw device 144, so as to enable collection of two separate blood samples (i.e., a paired blood draw) via the device 144. According to embodiments of the disclosure, the ports 214, 216 may be structured in accordance with any of the ports previously described for blood draw devices 14, 102, 104—with the ports 214, 216 provided as vacutainer ports, female luer connections, or female luer connections with integrated/bonded NFCs. The first port 214 is provided on housing 166 adjacent the distal end 178 thereof and is in fluid connection with a housing lumen 84 defined within the distal end 178 of housing 166, as shown in FIG. 9. The housing lumen 84 extends both distally and proximally from the location of a T-junction 90, with the lumen 84 extending distally through the coupling device 168 (e.g., through the blunted cannula 184) so as to be in fluid communication with the catheter assembly 142 (when blood draw device 144 is coupled to needleless access connector 162), and with the housing lumen 84 extending proximally so as to be joined to the inner volume 180 of housing 166.

According to embodiments of the disclosure, a split septum 92 may be provided in the housing lumen 84 at a location proximal from the T-junction 90 that may prevent a flow of fluid (i.e., blood) from the housing lumen 84 into the inner volume 180 of housing 166. In some embodiments, the split septum 92 is positioned within housing lumen 84 just proximal to the T-junction 90 and is formed of a flexible, elastomeric material with a slit formed therein through which the catheter tube 170 may pass when it is advanced from the first position to the second position.

With reference now to FIGS. 10 and 11, operation of blood draw device 144 to obtain first and second blood draws is shown and described herein in further detail. In an initial step, the blood draw device 144 is connected to a catheter assembly 142 that includes therein a catheter 148 that has previously been placed within the vasculature of a patient, so as to provide an indwelling catheter. As previously described, in one embodiment, the blood draw device 144 is connected to catheter assembly 142 via engagement of a coupling device 168 provided at the distal end 178 of housing 166 with a needleless access connector 162 of catheter assembly 142. Upon connection of coupling device 168 with the connector 162, the housing lumen 84 formed within housing 166 at distal end 178 is in fluid connection with the lumen within catheter connector 20.

Upon connection of blood draw device 144 to catheter assembly 142, a first vacutainer 218 (and associated access device, if necessary) is connected to the first port 214. Upon connection of the first vacutainer 218, the vacuum within first vacutainer 218 causes a flow of blood to commence through catheter assembly 142, through a portion of the housing lumen 84 in the distal end 178 of housing 166, and through the first port 214 into the first vacutainer 218—such that a first blood sample is collected therein. The first blood sample collected by first vacutainer 218 is obtained from a location within the vasculature of the patient where the distal tip 156 of indwelling catheter 148 is positioned. It is noted that, during collection of the first blood sample, the septum 92 in blood collection device 144 (which is positioned in main channel 86 proximal from the T-junction 90) prevents blood from flowing proximally into the inner volume 180 of housing 166, as shown in FIG. 9 with catheter tube 170 in the first position.

Upon collection of the first blood sample, the first vacutainer 218 may be disconnected from the first port 214 and the blood draw device 144 may then be operated to acquire a second blood sample. In preparing to acquire the second blood sample, the advancement member 172 of blood draw device 144 may be actuated (i.e., moved distally) to cause the catheter tube 170 to move from the first position, in which distal end 174 of catheter tube 170 is within housing 166 and proximal to the septum 92, to the second position, in which a distal end 174 of catheter tube 170 is advanced distally through septum 92 and out from housing 166 and further out distally past the distal end 156 of indwelling catheter 148, such as by a distance of 10 mm, as a non-limiting example.

Upon advancing of the catheter tube 170 to a desired position (i.e., with distal tip 174 of the catheter tube 170 extended distally past the distal tip 156 of indwelling catheter 148), the second vacutainer 220 is connected to the second port 216. Upon connection of the second vacutainer 220, the vacuum within second vacutainer 220 causes a flow of blood to commence through catheter tube 170 and through the second port 216 into the second vacutainer 220—such that a second blood sample is collected therein. The second blood sample collected by second vacutainer 220 is obtained from a location within the vasculature of the patient where the distal tip 174 of catheter tube 170 is positioned, which is different from the location at which the first blood sample was previously collected (at the distal tip 156 of indwelling catheter 148). It is noted that, during collection of the second blood sample, the advancement/positioning of catheter tube 170 through septum 92 and housing lumen 84 prevents blood from flowing proximally into the inner volume 180 of housing 166 and also prevents blood from flowing into/out the first port 214.

Beneficially, for each of the blood draw devices shown and described in FIGS. 1-11, the blood draw device provides for a blood draw to be performed from multiple sites through the indwelling catheter without additional vein puncture. Two separate blood samples may be obtained at two separate sites within the patient vasculature—with a first sample collected through the indwelling catheter at a location of the distal tip thereof and provided to a first vacutainer coupled to the blood draw device at a first port and a second sample collected through the advanced catheter tube at a location of the distal tip thereof (distal to the indwelling catheter) and provided to a second vacutainer coupled to the blood draw device at a second port. The two separate blood samples drawn from different locations may be used, for example, as a paired blood culture set (PBC) that may be tested for bacteremia and/or other contaminants, with the PBC providing a high microbiologic yield and an increased/improved sensitivity to detect pathogens and time to positivity (TTP), as compared to a single blood culture (SBC).

While exemplary catheter assemblies and blood draw devices are shown and described in FIGS. 1-11, it is recognized that embodiments of the invention encompass blood draw devices not described in detail herein. For example, distal and proximal ports (that provide for connection of vacutainers thereto) may be provided on blood draw devices that use other alternative advancement members or mechanisms, such as blood draw devices that include a turnable wheel on a housing thereof for advancing a catheter tube.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A blood draw device for use with a catheter assembly, the blood draw comprising:
a housing having a proximal end and a distal end, the housing defining a housing lumen at the distal end and defining an inner volume that is proximal to the housing lumen;
a coupling device positioned at the distal end of the housing and configured to mate with the catheter assembly;
a catheter tube movably received within the housing;
an advancement member movably coupled to the housing and configured to move relative to the housing to move the catheter tube between a first position, in which the catheter tube is disposed within the housing, and a second position, in which a distal end of the catheter tube is disposed beyond the distal end of the housing;
a first port provided on the housing at the distal end, the first port in fluid communication with the housing lumen;
a second port provided proximally from the housing, the second port in fluid communication with the catheter tube; and
a fluid flow preventing seal positioned in the housing lumen and proximal to the first port, wherein the distal end of the catheter tube is proximal to the fluid flow preventing seal when in the first position and extends through the fluid flow preventing seal when in the second position.

2. The blood draw device of claim 1, wherein the housing lumen comprises a main channel extending from the coupling device to the inner volume and a secondary channel extending from the main channel to the first port, the secondary channel joining to the main channel at a T-junction.

3. The blood draw device of claim 2, wherein the fluid flow preventing seal is positioned within the main channel proximal to the T-junction.

4. The blood draw device of claim 1, wherein the fluid flow preventing seal comprises a split septum, and wherein:
when the catheter tube is in the first position, the distal end of the catheter tube is positioned proximal to the split septum, such that split septum is closed and prevents fluid flow to the inner volume and to the catheter tube and the second port; and
when the catheter tube is in the second position, the catheter tube extends through the split septum.

5. The blood draw device of claim 1, wherein the coupling device comprises a male luer connector, the male luer connector including an elongated member and a rotating collar, the rotating collar configured to engage a connector of the catheter assembly in a threading fashion.

6. The blood draw device of claim 1, wherein the coupling device comprises a lock including a blunted cannula and a pair of locking arms for coupling to a connector of the catheter assembly.

7. The blood draw device of claim 1, wherein the advancement member comprises a telescopic cylinder having a proximal end and a distal end, with the distal end of the telescopic cylinder positioned within the inner volume and engaged with a proximal end of the catheter tube, and with the telescopic cylinder configured to move relative to the housing to move the catheter tube between the first position and the second position.

8. The blood draw device of claim 7, wherein the second port is connected to the proximal end of the telescopic cylinder, and wherein the telescopic cylinder includes a fluid path formed therethrough that fluidly connects the catheter tube to the second port.

9. The blood draw device of claim 1, wherein the advancement member comprises a first portion movably disposed along an upper surface of the housing and a second portion is movably disposed within the inner volume, with the advancement member seated within a slot formed in the upper surface of the housing and movable therethrough, and wherein a proximal end of the catheter tube is connected to the second portion.

10. The blood draw device of claim 9, further comprising a secondary catheter having a proximal end and a distal end, with the distal end of the secondary catheter coupled to the second portion and extending out proximally therefrom and out the proximal end of the housing, wherein the second port is connected to the proximal end of the secondary catheter and is fluidly connected to the catheter tube via the secondary catheter.

11. The blood draw device of claim 1, wherein each of the first port and the second port comprises a vacutainer port including:
an outer collar; and
a needle positioned within a circumference of the outer collar and secured to the housing, the needle comprising:
a needle lumen therein in fluid communication with the housing lumen; and
a needle tip positioned to pierce a seal of a vacutainer connected to the vacutainer port, thereby placing an inner chamber of the vacutainer in fluid communication with the needle lumen.

12. The blood draw device of claim 1, wherein each of the first port and the second port comprises a female luer connector, the female luer connector configured to couple with a vacutainer via an intervening luer lock access device.

13. The blood draw device of claim 12, wherein each of the first port and the second port further comprises a needle-free connector attached to the female luer connector, the needle-free connector including a septum therein configured to seal off the female luer connector, and wherein the needle-free connector is configured to couple with a vacutainer via an intervening luer lock access device.

14. The blood draw device of claim 13 wherein the needle-free connector is integrated with the female luer connector or solvent bonded to the female luer connector.

15. A method for acquiring a pair of blood samples comprising:
connecting a blood draw device to a catheter assembly including an indwelling catheter, the blood draw device comprising:
a housing having a proximal end and a distal end, the housing defining a housing lumen at the distal end and defining an inner volume that is proximal to the housing lumen;
a catheter tube movably received within the housing;
an advancement member movably coupled to the housing and configured to move relative to the housing to move the catheter tube between a first position, in which the catheter tube is disposed within the housing, and a second position, in which a distal end of the catheter tube is disposed beyond the distal end of the housing and beyond a distal end of the indwelling catheter;
a first port provided on the housing at the distal end, the first port in fluid communication with the housing lumen;
a second port provided proximally from the housing, the second port in fluid communication with the catheter tube; and
a fluid flow preventing seal positioned in the housing lumen and proximal to the first port, wherein the distal end of the catheter tube is proximal to the fluid flow preventing seal when in the first position and extends through the fluid flow preventing seal when in the second position;
connecting a first vacutainer to the first port with the catheter tube in the first position, with a first blood sample being drawn into the first vacutainer upon connection thereof to the first port, and wherein the fluid flow preventing seal is closed with the catheter tube in in the first position to prevent a flow of blood into the catheter tube and to second port;
actuating the advancement member to move the catheter tube to the second position; and
connecting a second vacutainer to the first port with the catheter tube in the second position, with a second blood sample being drawn into the second vacutainer upon connection thereof to the second port;
wherein the first blood sample is acquired from a first location in a patient vasculature adjacent the distal end of the indwelling catheter and the second first blood sample is acquired from a second location in the patient vasculature adjacent the distal end of the catheter tube.

16. The method of claim 15, wherein connecting the blood draw device to the catheter assembly comprises engaging a rotatable collar of a coupling device of the blood draw device to a connector of the catheter assembly.

17. The method of claim 15, wherein actuating the advancement member comprises actuating a telescopic cylinder distally within the housing to move the catheter tube to the second position, the second port connected to a proximal end of the telescopic cylinder.

18. The method of claim 15, wherein actuating the advancement member comprises distally actuating a first portion, in the form of a tab, along an upper surface of the housing, with the first portion joined to a second portion movably disposed within the inner volume, and wherein actuation of the first member causes the advancement member to move within a slot formed in the upper surface of the housing.

19. The method of claim 15, wherein connecting each of the first vacutainer to the first port and the second vacutainer to the second port comprises connecting a respective vacutainer to a vacutainer port comprising an outer collar and a needle positioned within a circumference of the outer collar, the needle including a needle lumen;
wherein, in connecting the respective vacutainer to the vacutainer port, the needle pierces a seal of the vacutainer to place an inner chamber of the vacutainer in fluid communication with the needle lumen and further in fluid communication with the housing lumen.

20. The method of claim 15, wherein connecting each of the first vacutainer to the first port and the second vacutainer to the second port comprises:
connecting a luer lock access device to a female luer connection of the respective port or a female luer connector of a needle-free connector attached to the respective port, the luer lock access device comprising a male luer connection and a needle including a needle lumen; and
connecting a respective vacutainer to the luer lock access device;
wherein, in connecting the respective vacutainer to the luer lock access device, the needle pierces a seal of the vacutainer to place an inner chamber of the vacutainer in fluid communication with the needle lumen and further in fluid communication with the housing lumen.
